(19) United States
(12) Patent Application Publication
(10) Pub. No.: US 2024/0285924 A1
(43) Pub. Date: Aug. 29, 2024

(54) Y-TYPE CONNECTOR

(71) Applicant: TOKAI MEDICAL PRODUCTS, INC., Aichi (JP)

(72) Inventors: Hironari KATO, Aichi (JP); Takao AKIOKA, Aichi (JP)

(73) Assignee: TOKAI MEDICAL PRODUCTS, INC., Aichi (JP)

(21) Appl. No.: 18/290,085

(22) PCT Filed: Mar. 24, 2022
(86) PCT No.: PCT/JP2022/013922

§ 371 Date: Nov. 9, 2023

(30) Foreign Application Priority Data
May 21, 2021 (JP) 2021-086217

(51) Int. Cl. A61M 39/10; A61M 25/01

(52) U.S. Cl. CPC A61M 39/10; A61M 25/01

(57) ABSTRACT

A Y-type connector includes a main tube portion and a side tube portion and allows a catheter or a guide wire to be smoothly inserted and removed with small resistance when the catheter or the guide wire is inserted from the side tube portion. A Y-type connector includes a main body, a sliding member, and a lid member, in which the main body has a sliding member insertion hole formed at least in a merging portion between a main tube passage portion and a side tube passage portion, the sliding member is made of a material having higher slidability than a material of a Y-type connector main body portion, and is formed to be insertable into the sliding member insertion hole, and the lid member is formed to be attachable to the main body in a state in which a sliding member is inserted into the sliding member insertion hole.

### TECHNICAL FIELD

The present invention relates to a Y-type connector.

### BACKGROUND ART

A Y-type connector is conventionally used to facilitate an operation of inserting or removing a guide wire or a catheter. As the Y-type connector, a Y connector has been proposed in which a cylindrical sub-branch for injecting a liquid medicine is branched from a cylindrical main branch through which a catheter is passed, the Y connector includes: a fixing valve inside the main branch, the fixing valve having a cylindrical shape with a hole that is reduced in diameter by being compressed in a longitudinal direction so as to fix the catheter passed through the hole; a first hemostasis valve and a second hemostasis valve capable of preventing outflow of blood by allowing the catheter to pass in an enclosed state; and further, an opener having a cylindrical shape movable in the longitudinal direction on a proximal end side of the main branch, in which only the first hemostasis valve is disposed within a moving range of the opener (Patent Literature 1).

However, in such a Y-type connector, when a guide wire or a catheter is inserted from the sub-branch side, the guide wire or the catheter is sharply curved at a portion merging with the main branch. This causes the problem that large resistance is generated at the time of insertion and removal of the guide wire or the catheter, and it is difficult to insert and remove the guide wire or the catheter.

### CITATIONS LIST

### Patent Literature

Patent Literature 1: JP 2005-261759 A

### SUMMARY OF INVENTION

### Technical Problems

Therefore, the present invention has been made to solve the above-described problem, and an object of the present invention is to provide a Y-type connector including a main tube portion having, in a longitudinal direction, a main tube passage portion through which an introduction member formed in a linear shape including a guide wire or a catheter is inserted, and a side tube portion extending in a side direction from the main tube portion, the Y-type connector allowing a catheter or a guide wire to be smoothly inserted and removed with small resistance when the catheter or the guide wire is inserted from the side tube portion.

### Solutions to Problems

The present invention has adopted the following means in order to achieve the above object.

A Y-type connector according to the present invention includes
a Y-type connector main body portion including a main tube portion having, in a longitudinal direction, a main tube passage portion through which an introduction member formed in a linear shape including a guide wire or a catheter is inserted, and a side tube portion having a side tube passage portion extending in a side direction from the main tube portion, wherein
the Y-type connector main body portion includes a main body, a sliding member, and a lid member,
the main body has a sliding member insertion hole formed at least in a merging portion between the main tube passage portion and the side tube passage portion,
the sliding member is made of a material having higher slidability than a material of the Y-type connector main body portion, or a material softer than the material of the Y-type connector main body portion, and formed in a form insertable into the sliding member insertion hole, and
the lid member is formed to be attachable to the main body in a state in which the sliding member is inserted into the sliding member insertion hole.

In the Y-type connector including the main tube portion and the side tube portion, after a long introduction member, such as a surgical guide wire and a catheter, is inserted from the side tube portion, the sliding member made of the material having higher slidability than that of the Y-type connector main body portion or the material softer than the material of the Y-type connector main body portion is inserted so as to be exposed to an inner surface of the side tube passage portion, which is a portion where resistance is likely to be generated because the introduction member is most likely to come into contact with the portion when pulled out, so that the resistance generated when the introduction member comes into contact can be reduced, and the introduction member can be smoothly pulled out. This prevents a material on the inner surface of the side tube passage portion from being scraped off by friction in a sliding portion.

In addition, in the Y-type connector according to the present invention, the sliding member may be formed larger than the sliding member insertion hole.

By adopting such a configuration, when the sliding member is covered with the lid member, the sliding member can exert a function as a gasket or a seal, and sealability can be secured without fixing the lid member by adhesion, welding, or the like.

Furthermore, in the Y-type connector according to the present invention, the sliding member may have a recessed groove formed in a portion disposed on an inner peripheral side of the Y-type connector main body portion.

By forming the groove in advance, a difference in level between the sliding member and the main tube passage portion or the side tube passage portion can be reduced, and the introduction member can be more smoothly inserted and removed.

Furthermore, in the Y-type connector according to the present invention, the lid member may include a piece extending in a direction substantially perpendicular to the longitudinal direction of the lid member at one side end portion, a fitting groove provided in the piece, and a protruding portion provided protruding in the longitudinal direction at another side end portion, and the main body may include a projection portion to be fitted into the fitting groove and a main body side groove to be fitted with the protruding portion.

By adopting such a configuration, the lid member can be positioned without using fixing means, such as adhesion and welding, for the lid member.

Furthermore, in the Y-type connector according to the present invention, a distal end side of the lid member may be formed in a tapered shape, and the Y-type connector may include a rotator having a contact surface capable of coming into surface contact with the tapered shape.

By adopting such a configuration, the lid member can be fixed while being pressed to a proximal end side and a central axis side.

Furthermore, in the Y-type connector according to the present invention, the lid member and the sliding member may be integrally formed.

By adopting such a configuration, the sliding member can be attached more easily.

### Advantageous Effects of Invention

In accordance with the Y-type connector according to the present invention, in a case in which the introduction member, such as a guide wire and a catheter, is inserted from the side tube portion side, the introduction member can be smoothly pulled out without resistance because the portion with which the introduction member comes into contact when pulled out has high slidability.

### BRIEF DESCRIPTION OF DRAWINGS

FIG. 1 is a perspective view of a Y-type connector 100 according to an embodiment.

FIG. 2 is a front view of the Y-type connector 100 according to the embodiment.

FIG. 3 is a cross-sectional view of the Y-type connector 100 according to the embodiment.

FIG. 4 is an exploded perspective view of the Y-type connector 100 according to the embodiment.

FIG. 5 is an exploded perspective view of the Y-type connector 100 according to the embodiment.

FIG. 6 is an exploded front view of the Y-type connector 100 according to the embodiment.

### DESCRIPTION OF EMBODIMENT

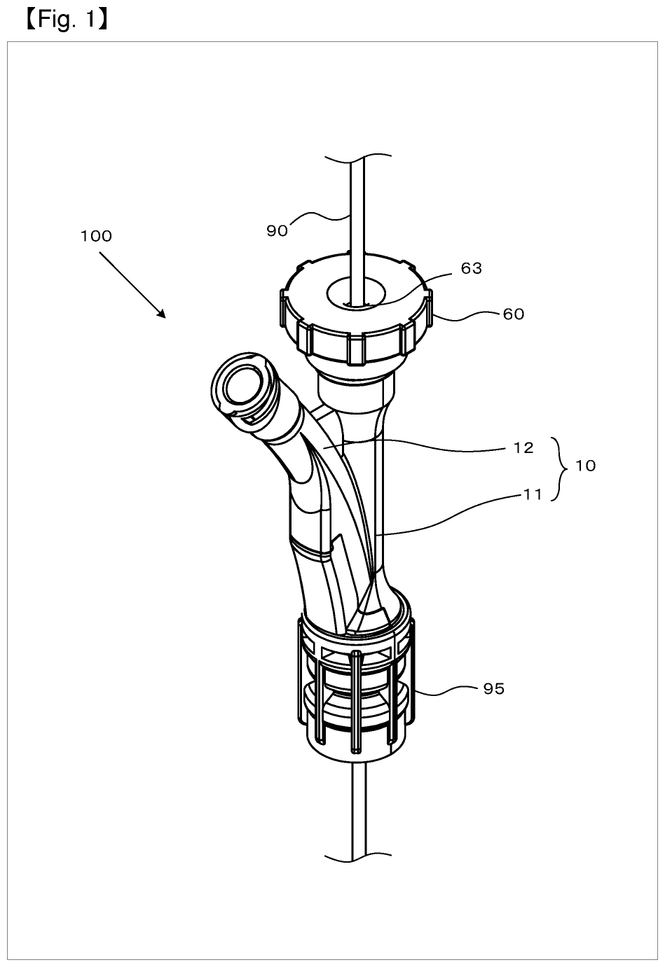

Next, an embodiment of a Y-type connector 100 according to the present invention will be described in detail with reference to the drawings. Note that the embodiment and drawings described below illustrate an example of embodiments of the present invention, are not used for the purpose of limiting the present invention to these configurations, and can be appropriately changed without departing from the gist of the present invention. For convenience of description, regarding the “proximal end side” and the “distal end side” in the claims and the specification, an upper side (near side) illustrated in FIG. 1 is referred to as “proximal end side”, and a lower side (far end side) is referred to as “distal end side” with respect to the Y-type connector 100.

### First Embodiment

As illustrated in FIGS. 1 to 3, the Y-type connector 100 according to a first embodiment includes a Y-type connector main body portion 10 mainly including a main tube portion 11 and a side tube portion 12, a fixing member 30 attached to the proximal end side of the Y-type connector main body portion 10, a screw cap 60, and a rotator 95.

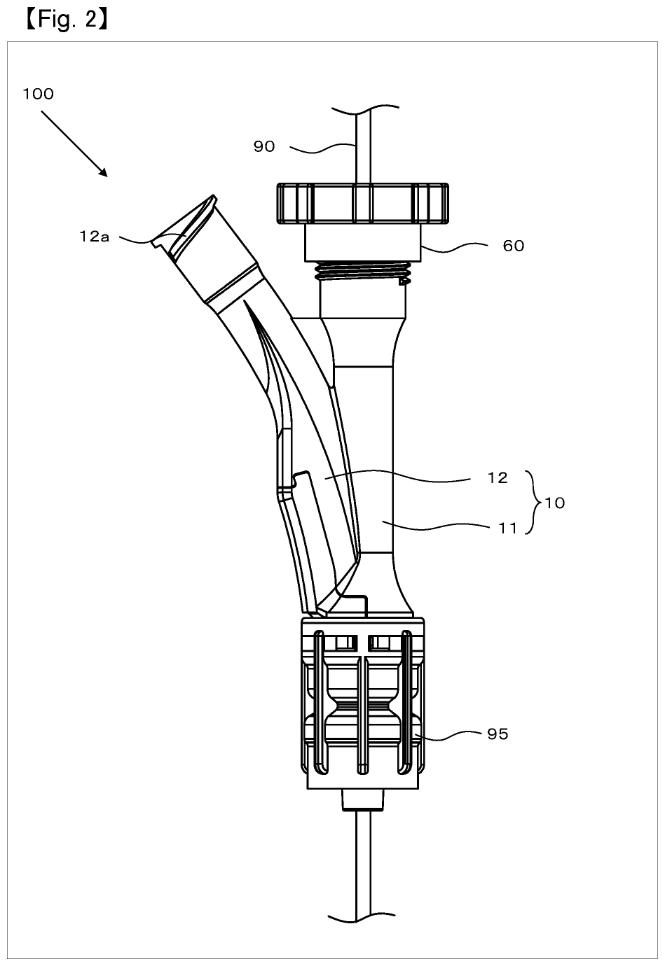

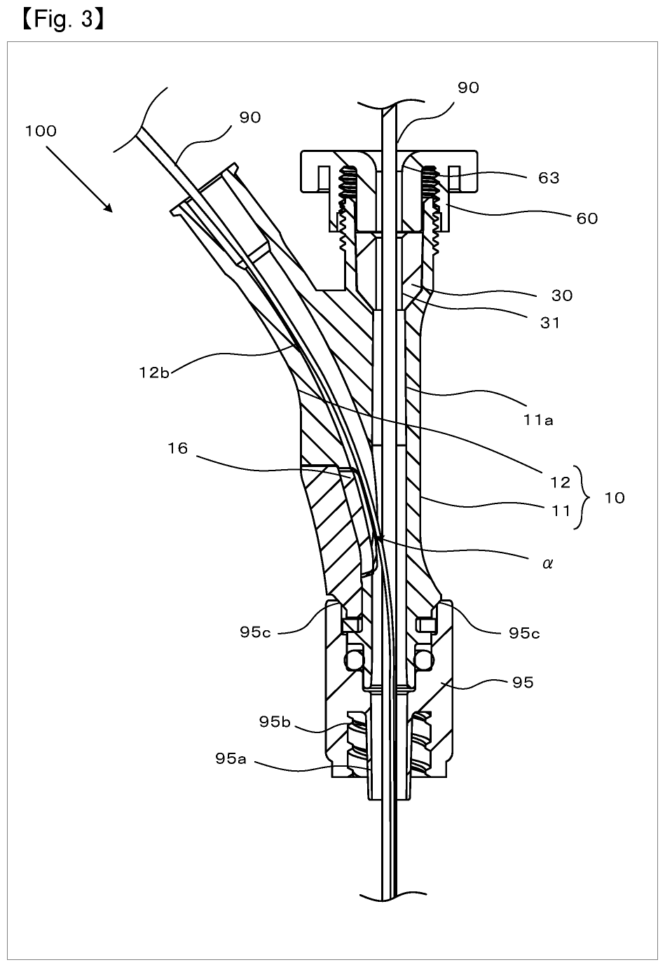

As illustrated in FIG. 1 or 3, the Y-type connector main body portion 10 includes the main tube portion 11 extending in a longitudinal direction from the proximal end side to the distal end side, and the side tube portion 12 extending in a side direction from the middle of the main tube portion 11. As illustrated in FIG. 3, the main tube portion 11 has a main tube passage portion 11a through which an introduction member 90 having an elongated linear shape, such as a guide wire and a catheter, is inserted and that passes through in the longitudinal direction. The fixing member 30 is housed in the main tube portion 11 on the proximal end side. As illustrated in FIG. 3, the side tube portion 12 is provided with a side tube passage portion 12b that communicates with the main tube passage portion 11a from an end portion of the side tube portion 12. The side tube portion 12 is used for injecting a medicinal solution, a contrast medium, or the like, or inserting an introduction member 90, such as a guide wire and a catheter, different from that of the main tube passage portion 11a. As illustrated in FIG. 2, a thread groove 12a is provided at an end portion of the side tube portion 12 such that an attachment tool such as a medicine injector can be connected.

As illustrated in FIG. 3, the fixing member 30 is used to fix the introduction member 90. The fixing member 30 is made of elastomer, rubber, silicon, or the like, and is formed in a cylindrical shape in which a through hole 31 through which the introduction member 90 is inserted is formed from the proximal end side to the distal end side. The fixing member 30 is pressed by the screw cap 60 from above to narrow the through hole 31, so that the introduction member 90 can be fastened and fixed.

The screw cap 60 has a function as a lid for housing the fixing member 30 in the Y-type connector main body portion 10, and also has a function of fastening and fixing the fixing member 30. The screw cap 60 is a cylindrical member in which a thread ridge is formed on its inner periphery so as to be engageable with a thread ridge formed on an outer periphery on the proximal end side of the Y-type connector main body portion 10, and can be rotated to compress or release the fixing member 30. As described above, by fastening the fixing member 30 by tightening the screw cap 60, the through hole 31 can be narrowed, and the introduction member 90 can be fastened and fixed. The screw cap 60 is provided with an introduction member hole 63 through which the introduction member 90 is inserted.

Furthermore, the rotator 95 is provided at a distal end of the Y-type connector main body portion 10. As illustrated in FIG. 3, the rotator 95 has: a through hole 95a through which the introduction member 90 is inserted and that passes through from the proximal end side to the distal end side; and a circular groove 95b formed around the through hole 95a. A guiding catheter or another catheter can be connected to the circular groove 95b.

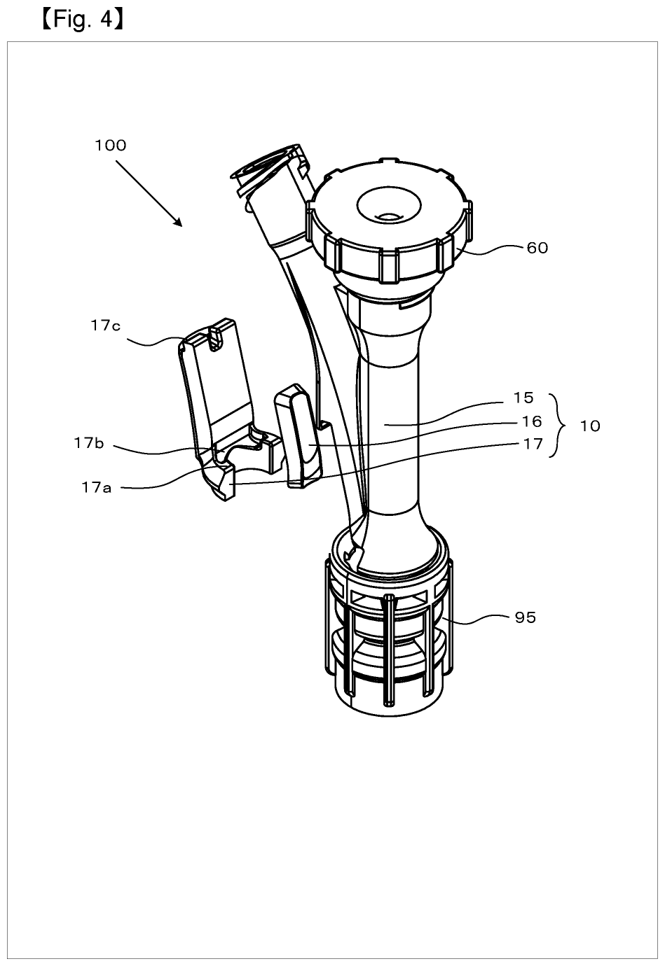

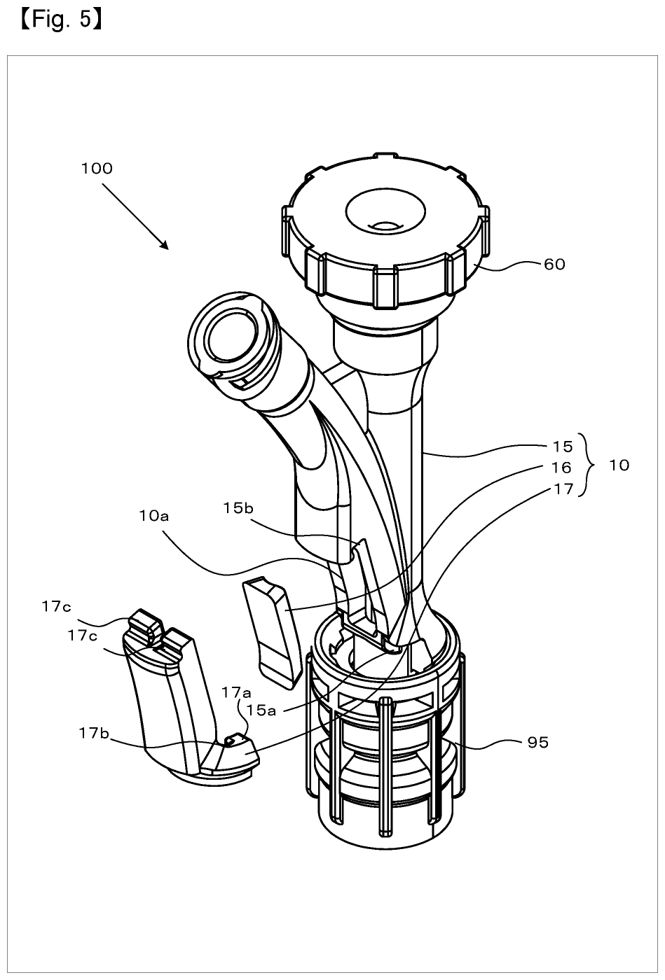

As illustrated in FIGS. 4 and 5, the Y-type connector main body portion 10 includes a main body 15, a sliding member 16, and a lid member 17. As illustrated in FIG. 3, the sliding member 16 is disposed inside a merging portion a between the main tube passage portion 11a and the side tube passage portion 12b of the Y-type connector main body portion 10. For the sliding member 16, a material having higher flexibility and higher slidability than a material of the Y-type connector main body portion 10, that is, a resin material having a low friction coefficient, or a material softer than the material of the Y-type connector main body portion is used. It has been found that even a material having lower slidability than the material of the Y-type connector main body portion 10 but softer than the material of the Y-type connector main body portion enables the introduction member 90 to be smoothly pulled out. Examples of the material used for the sliding member 16 include, but are not limited to, rubber materials such as silicon rubber and fluororubber, polyethylene, and various elastomers such as fluorine-based elastomers, polyurethane-based elastomers, polystyrene-based elastomers, and polyester-based elastomers. The sliding member 16 may be coated with a lubricant such as silicon oil, or the sliding member 16 may be a product in which a lubricant such as silicon oil is blended. The sliding member 16 is placed at a portion with which the introduction member 90, such as a guide wire and a microcatheter, inserted from the side tube passage portion 12b is particularly likely to come into contact when pulled out. Preferably, as illustrated in FIG. 3, the sliding member 16 is provided in the vicinity of the merging portion a between the main tube passage portion 11a and the side tube passage portion 12b so as to slightly protrude from an inner surface of the side tube passage portion 12b.

The main body 15, the sliding member 16, and the lid member 17 constituting the Y-type connector main body portion 10 are separately formed. As illustrated in FIG. 5, in the main body 15, a sliding member insertion hole 10a is formed in the merging portion a between the main tube passage portion 11a and the side tube passage portion 12b, and the sliding member 16 is disposed on inner peripheral surfaces of the main tube passage portion 11a and the side tube passage portion 12b by inserting the sliding member 16 into the sliding member insertion hole 10a from the outer peripheral side.

In the sliding member 16, a recessed groove 16a is formed in advance so as to conform to the inner peripheral surfaces of the respective passages when the sliding member 16 is disposed in the main tube passage portion 11a and the side tube passage portion 12b, so that the sliding member 16 easily conforms to the inner peripheral surfaces of the main tube passage portion 11a and the side tube passage portion 12b when inserted into the sliding member insertion hole 10a. In addition, in order to enable positioning of the sliding member 16 at a predetermined position of the sliding member insertion hole 10a, a side surface 16e is formed at an angle with respect to an inner peripheral surface or an outer peripheral surface such that an area 16d on the outer peripheral surface side is larger than an area 16c on the inner peripheral surface side. The sliding member 16 is formed slightly larger than the sliding member insertion hole 10a, and is formed to function as a seal or a gasket with pressure applied to the periphery when the lid member 17 is applying pressure and covering the sliding member 16.

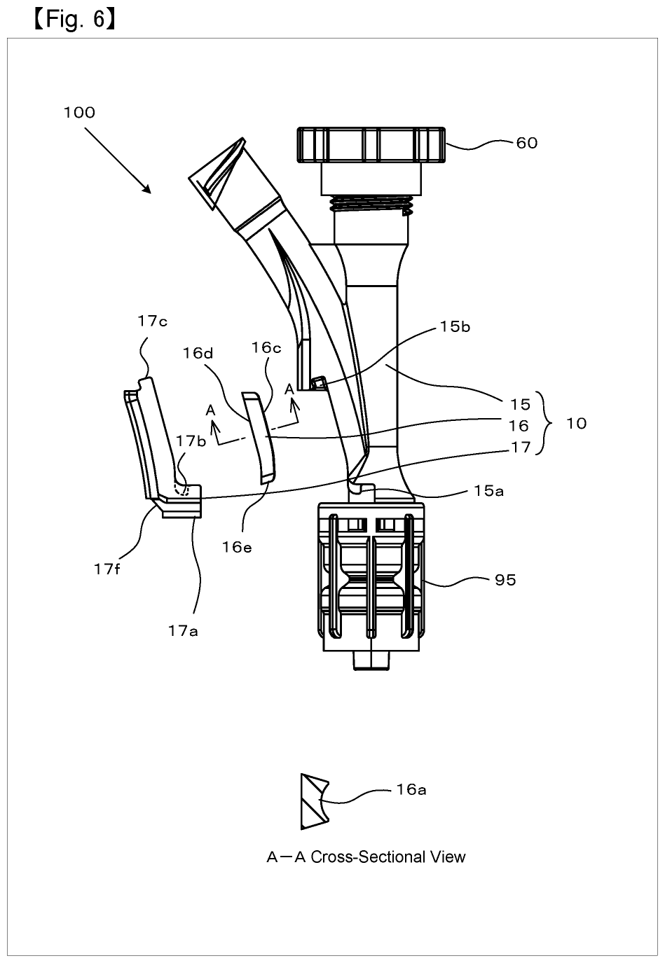

The lid member 17 is a member for fixing the sliding member 16 inserted into the sliding member insertion hole 10a. As a material, hard plastic may be used. Preferably, the same material as the main body 15 may be used. As illustrated in FIG. 5 or 6, the lid member 17 is provided with a piece 17a extending in a direction substantially perpendicular to the longitudinal direction of the lid member 17 at one side end portion, a fitting groove 17b provided in a distal end direction in the piece 17a, and a protruding portion 17c provided protruding in the longitudinal direction at another side end portion. On the other hand, the main body 15 is provided with a projection portion 15a to be fitted at a position corresponding to the fitting groove 17b, and a main body side groove 15b to be fitted at a position corresponding to the protruding portion 17c. In addition, an outer peripheral surface 17f of the lid member 17 on the rotator 95 side is obliquely tapered, and the rotator 95 has a contact surface 95c capable of coming into surface contact with this outer peripheral surface 17f formed in a tapered shape. Therefore, by attaching the rotator 95 in a state in which the fitting groove 17b and the projection portion 15a are fitted together and the protruding portion 17c and the main body side groove 15b are fitted together, the lid member 17 can be fixed so as to be pressed toward the proximal end side and the central axis side. At this time, as described above, since the sliding member 16 can function as a seal or a gasket when applied with pressure and covered by the lid member 17, it is possible to fix the lid member 17 with high sealability without using fixing means, such as adhesion and welding.

In the Y-type connector 100 formed as described above, the introduction member 90 inserted from the side tube portion 12 is pulled out while being in contact with the sliding member 16 when pulled out. Therefore, sliding resistance at the time of pulling out the introduction member 90 can be reduced, and the introduction member 90 can be smoothly pulled out. This prevents a material on the inner surface of the side tube passage portion from being scraped off by friction in a sliding portion. In addition, according to the Y-type connector 100 of the present embodiment, a user can appropriately select and use the material of the sliding member 16 from various sliding members 16 having different physical properties such as slidability, hardness, and transparency.

In the above-described embodiment, no fixing means, such as adhesion and welding, is used for the main body 15 and the lid member 17. However, in a case of providing stronger pressure resistance performance or the like, the main body 15 and the lid member 17 may be completely integrated by adhesion or welding.

Moreover, in the above-described embodiment, the sliding member 16 and the lid member 17 are completely separate parts, but the sliding member 16 and the lid member 17 may be integrated. In order to integrate the sliding member 16 and the lid member 17, the sliding member 16 and the lid member 17 may be separately formed and joined by adhesion or the like, or may be integrally formed at the time of forming by two-color molding.

Furthermore, in the above-described embodiment, the Y-type connector 100 including the fixing member 30 is used, but a Y-type connector including a hemostasis valve or a plunger may be adopted. In short, as long as the connector is a Y-type connector including the main tube portion 11 and the side tube portion 12, other configurations are not particularly limited.

### INDUSTRIAL APPLICABILITY

As described in the above embodiment, the Y-type connector can be used as an auxiliary tool in surgery using a guide wire or a catheter.

### REFERENCE SIGNS LIST

10 Y-type connector main body portion
10a Sliding member insertion hole
11 Main tube portion
11a Main tube passage portion
12 Side tube portion
12a Thread groove
12b Side tube passage portion
15 Main body
15a Projection portion
15b Main body side groove
16 Sliding member
16a Groove
16c Area
16d Area
16e Side surface
17 Lid member
17a Piece
17b Fitting groove
17c Protruding portion
17f Outer peripheral surface
30 Fixing member
31 Through hole
60 Screw cap
63 Introduction member hole
90 Introduction member
95 Rotator
95a Through hole
95b Circular groove
95c Contact surface
100 Y-type connector

1. A Y-type connector comprising
a Y-type connector main body portion including a main tube portion having, in a longitudinal direction, a main tube passage portion through which an introduction member formed in a linear shape including a guide wire or a catheter is inserted, and a side tube portion having a side tube passage portion extending in a side direction from the main tube portion, wherein
the Y-type connector main body portion includes a main body, a sliding member, and a lid member,
the main body has a sliding member insertion hole formed at least in a merging portion between the main tube passage portion and the side tube passage portion,
the sliding member is made of a material having higher slidability than a material of the Y-type connector main body portion, or a material softer than the material of the Y-type connector main body portion, and formed in a form insertable into the sliding member insertion hole, and
the lid member is formed to be attachable to the main body in a state in which the sliding member is inserted into the sliding member insertion hole.

2. The Y-type connector according to claim 1, wherein the sliding member is formed larger than the sliding member insertion hole.

3. The Y-type connector according to claim 1, wherein the sliding member has a recessed groove formed in a portion disposed on an inner peripheral side of the Y-type connector main body portion.

4. The Y-type connector according to claim 1, wherein
the lid member includes a piece extending in a direction substantially perpendicular to a longitudinal direction of the lid member at one side end portion, a fitting groove provided in the piece, and a protruding portion provided protruding in the longitudinal direction at another side end portion, and
the main body includes a projection portion to be fitted into the fitting groove and a main body side groove to be fitted with the protruding portion.

5. The Y-type connector according to claim 1, wherein a distal end side of the lid member is formed in a tapered shape, and the Y-type connector includes a rotator having a contact surface capable of coming into surface contact with the tapered shape.

6. The Y-type connector according to claim 1, wherein the lid member and the sliding member are integrally formed.
